(19) United States
(12) Patent Application Publication
(10) Pub. No.: US 2022/0017859 A1
(43) Pub. Date: Jan. 20, 2022

(54) Food Products Comprising Avian Stem Cells

(71) Applicant: VALNEVA SE, SAINT-HERBLAIN (FR)

(72) Inventors: Fabienne GUEHENNEUX, Le Temple De Bretagne (FR); Arnaud LEON, Nantes (FR); Brice MADELINE, Coueron (FR); Karine MOREAU, Chaumes en Retz (FR)

(21) Appl. No.: 17/296,000

(22) PCT Filed: Nov. 22, 2019
(86) PCT No.: PCT/EP2019/082218

§ 371 Date: May 21, 2021

(30) Foreign Application Priority Data
Nov. 23, 2018 (EP) 18208055.6

(51) Int. Cl. C12N 5/0735; A23J 1/02; A23K 10/10; A23K 10/20; A23L 13/50; A23L 33/17

(52) U.S. Cl. CPC C12N 5/0606; C12N 2500/32; A23K 10/10; A23K 10/20; A23L 13/50; A23L 33/17; A23V 2002/00; C12N 2500/14; C12N 2500/90; C12N 2501/125; C12N 2501/105; C12N 2501/115; C12N 2501/2306; C12N 2501/50; C12N 2501/13; A23J 1/02

(57) ABSTRACT

A synthetic meat product for human and animal consumption and methods for producing such food product are disclosed. The synthetic food product comprises or essentially consists of a cell biomass of avian cells grown in vitro in a chemically-defined serum free culture medium under controlled conditions and do not contain any hazard contaminations.

### FIELD OF THE INVENTION

The field of the present invention relates to industrial production of synthetic nutritive food products for human and/or animal consumption. More specifically, the invention relates to use of avian cell lines, particularly chicken or duck ES cell lines derived from stem cells of embryonic origin, for producing a cell biomass suitable as food or nutritional supplements. The invention encompasses the method of producing such synthetic food products and the products themselves.

### BACKGROUND OF THE INVENTION

Global meat production has increased rapidly over the past 50 year, i.e. total global production has grown 4-5 fold since 1961 (Ritchie and Roser, 2018). In 2014, total meat production was about 300 million tons, mostly poultry, pig and beef meat. Total livestock at the same time was about 1.4 billion cattle, 1.2 billion sheep, 1 billion goats, and about 1 billion pigs with a very strong increasing trend mainly driven by an increased Asian demand. Total meat consumption per capita has doubled during the last 50 years, i.e. meat consumption is higher than population increase. Furthermore, it is estimated that in 2030 the world meat consumption will increase by 25% as compare to 2015, and will reach 460 million tons in 2050 (GEAS 2012).

The other side of this impressive growth are serious problems associated with the current production of animal meat that will only further increase with the projected trend.

First, conventional methods of producing animal meat are highly inefficient. A significant portion of all agriculturally produced grain is used for animal consumption. Additionally, thousands pounds of water are required to produce one pound of meat. For example, production of one kilogram of pig, sheep/goat or bovine meat requires 5988, 8768 and 15415 liters of water, respectively (Mekonnen and Hoekstra, 2010). Despite that, present efforts are focused on fastening livestock growth by using hormones and antibiotics and thus consuming less grain and water. However, this development leads to another problem where the livestock meat contaminated with growth hormones (especially, steroid hormones, such as testosterone, progesterone, estrogen, or their synthetic derivatives) and antibiotics is a threat to public health (Galbraith, 2002; Jeong et al., 2010).

Second, the intensification of livestock farming is associated with a quick spread of pathogens and emerging diseases throughout the world (Greger, 2007). Such food borne pathogens like Salmonella, Campylobacter and Escherichia coli, are responsible for millions of episodes of illness each year and cause massive expenditures in the human and animal health systems.

Third, huge emissions of carbon dioxide and methane from the livestock production sector is a serious environmental problem (GEAS 2012; Opio et al., 2013; Hedenus et al., 2014). The World Bank estimates that 18% of global CO₂ emissions are caused by the current ineffective meat production. The Worldwatch Institute claims that the true figure is 51% (see https://www.independent.co.uk/environment/climate-change/study-claims-meat-creates-half-of-all-greenhouse-gases-1812909.html).

Forth, the current methods to produce meat involve the suffering of animals that many people object to nowadays.

Fifth, an additional disadvantage of using natural meat for consumption is related to high content of harmful substances, such as cholesterol and saturated fat that cause some dietary and health-threatening issues.

Thus, there is a need of developing new approaches for production of meat and/or meat-like products that can at least partly solve or reduce the above-mentioned issues.

One approach can be to develop nontraditional meat products generated ex vivo. The so-called “synthetic or “in vitro meat”, also known as “cell-cultured meat”, “artificial meat”, “clean meat” or “lab-grown meat”, is manufactured by using cells cultured in vitro and originally derived from animals. Such synthetic meat has a number of advantageous relative to conventional meat in terms of efficiency of natural resource (land, energy, water) use, lower greenhouse gas production and better animal welfare (Tuomisto, 2014). Furthermore, the nutrient composition of cultured meat can be thoroughly controlled, thereby avoiding contamination with hazard components, such as cholesterol, saturated fat, hormones, antibiotics and infectious microorganisms.

In theory, the synthetic meat could play a complementary role alongside conventional meat products, or even could be seen as an alternative to meat, provided that the physical properties, colour, flavour, aroma, texture, palatability and nutritional value would be comparable to traditional animal meat or simply would be acceptable to humans. Even though some progress has been made during recent years, technologies in the area of synthetic meat or meat-like production are still at a very early stage of implementation (reviewed in Kadim et al., 2015). Important issues remained to be resolved including the choice of the appropriate cell types, perfection of culture conditions and development of culture media that are cost-effective and free of hazard contaminants.

One important issue that is among others solved by the present invention is the scale up in order to produce massive amounts of meat like products at a reasonable price.

The present inventors have developed avian cell lines that can persistently grow in culture and produce a large cell biomass. In particular, the cell lines presented herein have all characteristics required to make a high industrial scale culture feasible.

### SUMMARY OF THE INVENTION

There is a high need to find alternative methods to produce food products that are free of antibiotics and require less energy and water. Unexpectedly, culturing avian cell lines in suspension provided an extremely high yield source for such food products.

The present application provides a new process for producing synthetic meat products that could help solving serious environment, health and ethical problems associated with the traditional approaches and satisfy rapidly growing consumers' needs. The disclosed process does not involve a cumbersome procedure of tissue engineering but it is based on a low cost cell culture. Aspects of the invention provide, in particular, the following:

A1. A process/method of “in vitro” producing a nutritive food product for human or animal consumption comprising culturing an avian cell line in suspension, wherein said avian cell line is i) derived from avian embryonic stem cells, ii) capable of proliferating in a basal culture medium in the absence of exogenous growth factors, feeder cells and/or animal serum, and iii) capable of growing continuously in suspension.

A2. The process/method of aspect A1, wherein the avian cell line is obtained by the process comprising the steps:
a) isolating avian embryonic stem cells from an embryo(s) at a developmental stage around oviposition;
b) culturing said cells in a basal culture medium containing at least one exogenous growth factors SCF, IGF-1, bFGF, IL-6, IL-6R and/or CNTF, a layer of feeder cells and an animal serum for at least twenty passages;
c) modifying said culture medium by progressive deprivation of said growth factors, feeder cells and animal serum and further culturing the cells for at least several passages; and
d) adapting the cells of step c) to suspension,
   thereby obtaining the established avian cell line capable of proliferating in a basal culture medium in the absence of exogenous growth factors, feeder cells and/or animal serum for at least 50 days.

A3. The process/method of aspects A1 and A2, wherein the avian cell line is obtained by the process comprising the steps:
a) isolating the avian embryonic stem cells from an embryo(s) at a developmental stage around oviposition;
b) culturing said cells in a basal culture medium containing the exogenous growth factor IGF-1 and CNTF, a layer of feeder cells and an animal serum for at least one passage;
c) progressively withdrawing said growth factors from the culture of step b) and growing for at least one passage;
d) progressively withdrawing the feeder cells from the culture of step c) and growing for at least one passage;
e) progressively withdrawing the animal serum from the culture of step d) and growing for at least one passage; and
f) adapting the cells of step e) to suspension,
   thereby obtaining the continuous avian cell line capable of proliferating in a basal medium in the absence of exogenous growth factors, feeder cells and/or animal serum.

A4. The process/method of any of aspects A1 to A3, wherein the avian cell line is derived from a chicken embryonic stem cell.

A5. The process/method of any of aspects A1 to A4, wherein the avian cell line is derived from a duck embryonic stem cell.

A6. The process/method of any of aspects A1 to A5, wherein the avian cell line is free of functional endogenous retroviral or other viral particles.

A7. The process/method of any of aspects A1 to A6, wherein the avian cell line is derived from a SPF specie.

A8. The process/method of any of aspects A1 to A7, wherein the avian cell line is selected from the group consisting of the chicken EB14, chicken EB line 0, chicken EBv13, chicken DL43, chicken DL46, duck EB24, duck EB26 and duck EB66 cell lines.

A9. The process/method of any of aspects A1 to A8, wherein the avian cell line is the chicken DL43, chicken DL46, duck EB24, duck EB26.

A10. The process/method of any of aspects A1 to A9, wherein the avian cell line is the chicken DL43 or duck EB26.

A11. The process/method of any of aspects A1 to A10, wherein the cell line is grown in a culture medium, which is a synthetic or chemically defined (CD) medium free of hazardous substances for humans and/or animals.

A12. The process/method of aspects A11, wherein the synthetic medium is Ex-Cell® GRO-I and/or HYQ CDM4 Avian medium.

A13. The process/method of aspect A11, wherein the synthetic or CD medium is additionally supplemented with one or more ingredient(s) selected from the group consisting of amino acids, nucleotides, vitamins, saccharides, fatty acids, beta-mercapto-ethanol, insulin, glycine, choline, pluronic acid F-68 and sodium pyruvate.

A14. The process/method of aspect A13, wherein the additional ingredient is L-glutamine used at a concentration from 0 to 12 or from 1 to 5 mM, preferably about 2.5 mM.

A15. The process/method of any of aspects A11 to A13, wherein the culture medium further contains plant and/or yeast hydrolysates.

A16. The process/method of any of aspects A11 to A15, wherein the culture medium is free of any animal product, including serum.

A17. The process/method of any of aspects A1 to A16, wherein the cell line is cultured under fed-batch conditions.

A18. The process of any of aspects A1 to A16, wherein the cell line is cultured under perfusion conditions.

A19. The process/method of any of aspects A1 to A18, wherein the cells is cultured in a bioreactor with a volume equal or larger than 30 liters, 50 liters, 100 liters, 1000 liters, preferably 10,000 liters.

A20. The process/method of any of aspects A1 to A19, wherein the cell line is cultured at a temperature around 37° C., pH 7.2, pO2 about 50%, and with the stirring speed of about 40 rpm or higher.

A21. The process/method of any of aspects A1 to A20, wherein the cell line is cultured until the cell density has reached about 10⁷ cells/mL.

A22. The process/method of any of aspects A1 to A21, wherein the cell is cultured until the cell density has reached about 10⁸ cells/mL.

A23. The process/method of any of aspects A1 to A21, wherein the cell line is cultured until the cell density has reached more than 10⁸ cells/mL.

A24. The process/method of any of aspects A1 to A23, wherein the yield of the process is at least about 0.5 to 1 g biomass per g medium.

A25. The process/method of any of aspects A1 to A24, further comprising a step of cell biomass harvesting by sedimentation and decantation.

A26. The process/method of aspect A25, wherein cell sedimentation is performed by addition of a calcium salt to cell suspension.

A27. The process/method of aspect A26, wherein a calcium salt is calcium chloride used at a final concentration from 10 to 500 mg/L, preferably from 50 to 300 mg/L, more preferably 50 mg/L.

A28. The process/method of any of aspects A1 to A27, further comprising a step of adding to the cell biomass one or more ingredient(s) increasing nutritional value of the food product selected from the group comprising vitamins, co-vitamins, minerals, essential amino acids, essential fatty acids, enzymes and antioxidants.

A29. The process/method of any of aspects A1 to A28, further comprising adding to the cell biomass one or more flavorant(s), flavor aromatic(s) and/or colorant(s).

A30. The process/method of any of aspects A1 to A29, further comprising one or more a food processing step(s) selected from cooling, freezing, solidifying, drying, pickling, boiling, cooking, baking, frying, smoking, 3D printing and packing.

B1. A food product produced by the process/method of any of aspects A1 to A30.

C1. A cell biomass produced by the process/method of any of aspects A1 to A27.

D1. Use of the cell biomass of aspect C1 for producing a synthetic food product for human or animal consumption.

B2. A food product comprising or essentially consisting of the cell biomass of aspect C1.

B3. The food product of aspects B1 or B2, further comprising other cells such as non-human muscle cells, fat cells or cartilage cells, or their combinations, that are grown in vitro together with the avian cells or added after the avian cells harvesting.

B4. The food product of any of aspects B1, B2 or B3, further comprising additional ingredients enhancing the nutritional value selected from the group comprising minerals, vitamins, co-vitamins, essential fatty acids, essential amino acids, enzymes and antioxidants, or their combinations.

B5. The food product of any of aspects B1, B2 to B4, further comprising one or more flavorant(s), flavor aromatic(s) and/or colorant(s), or their combinations.

B6. The food product of any of aspects B1, B2 to B5, wherein the food product is processed to any of the consumption form selected from the group comprising paste, puree, soup, pie, powder, granules, chip, tablet, capsule, spread and sausage.

### BRIEF DESCRIPTION OF THE DRAWINGS

The present invention is further illustrated by the following Figures, Tables and Examples from which further features, embodiments and advantages may be taken. As such, the specific modifications discussed are not to be construed as limitations on the scope of the invention. It will be apparent to the person skilled in the art that various equivalents, changes, and modifications may be made without departing from the scope of the invention, and it is thus to be understood that such equivalent embodiments are to be included herein.

In connection with the present invention

FIG. 1: Process of avian stem cells adaptation to CDM4 avian medium before banking.

FIG. 2: Cell growth parameters along avian stem cells adaptation in CD medium. (A) Cell density (solid line) and viability (dashed line). (B) Population Doubling Time (PDT).

FIG. 3: Cell recovery post thawing. (A) Cell density (solid line) and viability (dashed line) after thawing. (B) Population Doubling Time (PDT).

FIG. 4: Growth kinetics of avian stem cells seeded at different concentrations. (A) Cell density obtained after 3 or 4 days of culture in CD medium supplemented with 2.5 mM L-glutamine. (B) Percentage of viability obtained at Day 3 and Day 4 after seeding at the different concentrations.

FIG. 5: Scale-up process to amplify cells for the seeding of the 30 L bioreactor.

FIG. 6: Typical densities obtained during the avian stem cells scale-up. Cell density (solid line) and viability (dashed line) of avian stem cells during the amplification process.

FIG. 7: Cell growth of CD adapted avian stem cells in 30 L bioreactor. Cell density (solid lines) and viability (dashed lines) of avian stem cells during the amplification process in bioreactor.

FIG. 8: 1 L-bottles containing cell suspension harvested from 30 L bioreactor.

FIG. 9: 500 mL-tubes containing an avian stem cell pellet produced in 30 L bioreactor.

### DETAILED DESCRIPTION OF THE INVENTION

It was recognized by the inventors that meat of domestic birds, especially chicken and duck meat, is a major source of comestible protein. It was also recognized that traditional approaches of producing poultry meat or meat in general are neither efficient nor produce a healthy product in amounts sufficient to cover the rapidly growing consumers' needs and growing numbers of meat consumers.

In vitro grown “poultry” food could be an alternative conventionally produced poultry meat or a supplement to food products. Importantly, in vitro culturing is performed under controlled sterile conditions, thereby allowing generation of synthetic food products free of harmful contaminations. Additionally, the herein described culture processes are suitable for producing a cell biomass at industrial scale for a reasonable price.

Therefore, an objective of the present invention is to provide a food product produced from avian cells grown in vitro, which can be used as a substitution of a conventional chicken or duck meat, or any meat or a supplement to synthetic meat products.

In one aspect, the present application provides a method for producing a synthetic food product cultured in vitro.

The term “synthetic food product” refers to a product produced in culture of cells isolated from non-human animals, which is useful for consumption. The term “synthetic food product”, as used herein, is interchangeable with such terms as “meat-like product”, “synthetic meat”, “in vitro meat”, “cultured meat”, “cell-cultured meat”, “clean meat”, “artificial meat” and “lab-grown meat”.

By “in vitro” it is meant that the process is carried out on isolated cells outside of the living organism, particularly on isolated cells grown in a synthetic culture medium.

### Avian Cell Line

In one embodiment, the method of the present invention is conducted, but not exclusively, on an avian cell line. The term “avian” or “bird” refer to any species, subspecies or race of organism of the taxonomic class “ava”. More specifically, “birds” refer to any animal of the taxonomic order Anseriformes (duck, goose, swan and allies), Galliformes (chicken, quails, turkey, pheasant and allies) and Columbiformes (pigeon and allies).

In one embodiment, the bird is selected among specific-pathogen-free (SPF) species that do not produce infectious endogenous retrovirus particles. “Endogenous retrovirus particle” means a retroviral particle or retrovirus encoded by and/or expressed from ALV-E or EAV proviral sequence present in some avian cell genomes. For instance, ALV-E proviral sequences are known to be present in the genome of domestic chicken (except Line-0 chicken), red jungle fowl and Ringneck Pheasant. EAV proviral sequences are known to be present in all genus gallus that includes domestic chicken, Line-0 chicken, red jungle fowl, green jungle fowl, grey jungle fowl, Ceylonese jungle fowl and allies (see Resnick et al., 1990). Therefore, preferably the bird is selected from the group comprising ducks, gooses, swans, turkeys, quails, Japanese quail, Guinea fowl, Pea Fowl, which do not produce infectious endogenous ALV-E and/or EAV particles.

In a preferred embodiment, the bird is a chicken, especially, the chicken from the genus Gallus. For instance, the chicken strain is selected among ev-0 domestic chicken species (Gallus Gallus subspecies domesticus), especially from the strains ELL-0, DE or PE11. In another preferred embodiment, the chicken is selected from SPF species screened for the absence of reticuloendotheliosis virus (REV) and avian exogenous leucosis virus (ALV-A, ALV-B, ALV-C, ALV-D or ALVA, especially from White Leghorn strain, most preferably from Valo strain.

In another preferred embodiment, the bird is a duck, more preferably, the domestic Pekin or Muscovy duck, most preferably, Pekin duck strain M14 or GL30.

In yet one embodiment, the cell line of the invention is derived from avian pluripotent embryonic stem (ES) cells. By “pluripotent” is meant that the cells are non-differentiated or the cells are capable of giving rise to several different cell types, e.g. muscle cells, fat cells, bone cells or cartilage cells but are not capable of developing into a whole living organism. Preferably, the avian pluripotent ES cells are obtained from avian embryo(s), especially at a very early development stage, e.g. at blastula stage. More specifically, the ES cells are isolated from the embryo around oviposition, e.g. before oviposition, at oviposition, or after oviposition. Preferably, the ES cells are isolated from the embryo at oviposition. A man skilled in the art is able to define the timeframe prior egg laying that allows collecting appropriate cells (see Sellier et al., 2006; Eyal-Giladi and Kochan, 1976).

Alternatively, the avian cell line may be derived from totipotent ES cells, such as cells from the blastocyst stage of fertilized eggs.

Alternatively, the ES cell line may be obtained from Primordial Germ Cells (PGCs). For instance, PGCs may be isolated from embryonic blood collected from the dorsal aorta of a chicken embryo at stage 12-14 of Hamburger & Hamilton's classification (Hamburger & Hamilton, 1951). Otherwise, PGCs may be collected from the germinal crescent by mechanical dissection of avian embryo or from the gonads (see, e.g. Chang et al., 1992; Yasuda et al., 1992; Naito et al., 1994).

Additionally, the avian cell line of the invention may be derived from avian induced Pluripotent Stem cells (iPSCs).

Yet alternatively, the avian cell line of the invention may be derived from avian somatic stem cells.

In another embodiment, the avian cell line of the invention can serve as precursor cells to obtain partially differentiated or differentiated cells. Indeed, these stem cells are pluripotent, meaning that they have the potential to be induced in multiple differentiation pathways, in particular, conversion into muscle cells, or fat cells, or cartilage cells, or other appropriate cells.

In yet one embodiment, the avian cell line is a continuous cell line. Under “continuous” it is meant that the cells are able to replicate in culture over an extended period of time. More specifically, the cells of the invention are capable of proliferating in a culture for at least 50 days, at least 75 days, at least 100 days, at least 125 days, at least 150 days, at least 175 days, at least 200 days, at least 250 days or indefinitely.

In yet one embodiment, the avian cell line, such as e.g. a duck or chicken cell line, is continuous and stable. Under “stable” it is meant that the cells have a stable cell cycle duration conducting to a stable population doubling time and controlled proliferation, stable phenotype (shape, size, ultrastructure, nucleocytoplasmic ratio), stable optimal density, when maintained in defined conditions, and stable expression of proteins (such as, for example, telomerase) and markers (such as, for example, SSEA1 and EMA-1). In a preferred embodiment, the avian cell line, in particular, the EBx cell line, has a stable phenotype (shape, size, ultrastructure, nucleocytoplasmic ratio) characterized in high nucleo-cytoplasmic ratio, high telomerase activity and expression of one or more ES cell markers, such as alkaline phosphatase and SSEA-1, EMA-1 and ENS1 epitopes, and has a stable cell cycle. These parameters can be measured by techniques well known in the art. For instance, the stable phenotype can be measured by electronic microscopy. The cell cycle can be measured based on monitoring of the DNA content by flow cytometry using a co-staining with BromoDeoxyuridine (BrDU) and Propidium Iodide (PI). The skilled person in the art may also use other methods.

In one more embodiment, the cell line of the present invention is genetically stable meaning that all cells maintain similar karyotype along passages.

Preferably, the avian ES cells of the invention do not undergo any specifically introduced genetic modification to replicate indefinitely. The continuous cell line may be derived spontaneously following a multi-step process permitting the selection of stable cells that maintain some of the unique biological properties of ES cells, such as the expression of ES cell specific markers (e.g., telomerase, SSEA-1, EMA-1), the ability to indefinitely self-renew in vitro and a long-term genetic stability (Olivier et al., 2010; Biswas and Hutchins, 2007).

Alternatively, the continuous cell phenotype can be obtained by genetic modifications and/or a process of immortalization. By “immortalization” it is meant that the cells, which would normally not proliferate indefinitely but, due to mutation(s), have evaded normal cellular senescence and can keep undergoing division. The mutation(s) may be induced intentionally, e.g. by physical, chemical or genetic modification. Physical modification may be achieved by UV-, X-ray or gamma-irradiation. Chemical modification may be achieved by chemical mutagens (substances, which damage DNA). By genetic modification it is meant that the cells may be transiently or stably transfected with virus or non-viral vector, for gene overexpression, e.g proto-oncogenes, telomerase or transcriptional factors, such as OCT4, Klf4, Myc, Nanog, LIN28, etc. Methods of immortalization of cells are described, for instance, in the patent applications: WO2009137146 (quail cells immortalized with UV-light), WO2005042728 (duck cells immortalized by viral transfection), and WO2009004016 (duck cells transfected with non-viral vector), incorporated herein by reference in their entirety.

In one more embodiment, the avian cell line of the present invention is a non-adherent cell line meaning that the cells can grow in suspension without any support surface or matrix. The cells of the invention may become non-adherent spontaneously during culturing or the non-adherence is obtained by withdrawal of the feeder layer. The non-adherent cells can proliferate in culture suspension for an extended period of time until high cell densities are reached. Therefore, they are perfectly suitable for large-scale manufacturing in bioreactors.

Additionally, the cells of the invention has at least one of the following characteristics: a large nucleus, a high nucleo-cytoplasmic ratio, a stable number of chromosomes, elevated telomerase activity, positive alkaline phosphatase activity and expression of EMA1, ENS1 and SSEA-1 surface epitopes (ES-specific markers). Alternatively, these cells may be genetically modified so, as to produce a substance of interest, e.g. a protein, lipid, enzyme, vitamin, etc.

In one embodiment, the avian cell line of the present invention is obtained by the methods previously described in WO2003076601, WO2005007840 or WO2008129058 incorporated herein by reference in their entirely. Briefly, the avian ES cells are isolated from bird embryo(s) around oviposition. The cells are cultured in a basal culture medium containing all factors to support cell growth, additionally supplemented with at least one, preferably two growth factors such as Insulin Growth factor 1 (IGF-1), Ciliary Neurotrophic Factor (CNTF), Interleukin 6 (IL-6), Interleukin 6 Receptor (IL-6R), Stem Cell Factor (SCF) and/or Fibroblast Growth Factor (FGF), animal serum and feeder layer cells. After several passages, the culture medium is modified progressively by decreasing and/or completely withdrawing growth factors, animal serum and feeder layer cells, followed by further adaption of cells to suspension. This gradual adaptation of cultured cells to the basal synthetic medium results in obtaining adherent or non-adherent avian cell lines (herein referred to also as “EBx” or “EBx cell line(s)”), which are capable to proliferate in culture for a long time, especially for at least 50 days, at least 250 days, preferably indefinitely. The established EBx cell lines can grow in suspension in a basal culture medium, free of exogenous growth factors, animal serum and feeder layer cells, for at least 50 days, 100 days, 150 days, 300 days or 600 days.

More specifically, the avian cell line may be obtained by the process comprising the steps:
a) isolating avian embryonic stem cells from an embryo(s) at a developmental stage around oviposition;
b) culturing said cells in a basal culture medium containing at least one exogenous growth factor SCF, IGF-1, bFGF, IL-6, IL-6R and CNTF, a layer of feeder cells and an animal serum for at least twenty passages;
c) modifying said culture medium by progressive deprivation of said growth factors, feeder cells and animal serum and further culturing the cells for at least several passages; and
d) adapting the cells of step c) to suspension,
   thereby obtaining the established cell line capable of proliferating in a basal culture medium in the absence of exogenous growth factors, feeder cells and/or animal serum for at least 50 days, preferably at least 600 days.

Alternatively, the avian cell line may be obtained by the process comprising the steps:
a) isolating the avian embryonic stem cells from an embryo(s) at a developmental stage around oviposition;
b) culturing said cells in a basal culture medium containing the exogenous growth factor IGF-1 and CNTF, a layer of feeder cells and an animal serum for at least one passage;
c) progressively withdrawing said growth factors from the culture of step b) and further growing for at least one passage;
d) progressively withdrawing the feeder cells from the culture of step c) and further growing for at least one passage;
e) progressively withdrawing the animal serum from the culture of step d) and further growing for at least one passage; and
f) adapting the cells of step e) to suspension,
   thereby obtaining the established avian cell line capable of proliferating in a basal medium in the absence of exogenous growth factors, feeder cells and/or animal serum for a long period (at least 50 days), preferably indefinitely.

By “passage” it is meant the transfer of cells, with or without dilution, from one culture vessel to another. This term is synonymous with the term ‘sub-culture’. The passage number is the number of times the cells are sub-cultured or passed in a new vessel. This term is not synonymous with a population doubling time (PTD) or generation which is the time needed by a cell population to replicate one time. For example, isolated avian ES cells of step a) of the process described above have the PDT of around >40 hours. The cells of the established avian cell line have the PDT of around <30 hours or around <20 hours. For ES cells one passage usually occurs every 3 generations.

By “progressive deprivation or withdrawing”, it is meant a gradual reduction of any component up to its complete disappearance (total withdraw) spread out over time. For the establishment of the cell line of the present invention, the withdrawal of growth factors, serum and/or feeder layer leads to the isolation of populations of avian embryonic derived stem cells, which can grow indefinitely in basic culture media.

By “adapting to suspension”, it is meant adapting cells to grow as non-adherent cells without any supportive surface, matrix or carrier.

According to the invention, “basal culture medium” means a culture medium with a classical media formulation that allows, by itself, at least cells survival, and even better, cell growth. Preferably, the basal medium is a synthetic or chemically defined (CD) medium. Such medium comprises inorganic salts (e.g. CaCl₂, KCl, NaCl, NaHCO₃, NaH₂PO₄, MgSO₄), amino acids (e.g., L-Glutamine), vitamins (e.g., thiamine, riboflavin, folic acid, D-Ca panthothenate) and optionally others components such as glucose, sucrose, beta-mercapto-ethanol and sodium pyruvate. Non-limiting examples of basal media are SAFC Excell media, BME (basal Eagle Medium), MEM (minimum Eagle Medium), medium 199, DMEM (Dulbecco's modified Eagle Medium), GMEM (Glasgow modified Eagle medium), DMEM-HamF12, Ham-F12 (Gibco) and Ham-F10 (Gibco), IMDM (Iscove's Modified Dulbecco's medium), MacCoy's 5A medium, RPMI 1640, and GTM3.

In some embodiments, the basal synthetic medium may be supplemented with at least one growth factors selected from the group comprising IL-6, IL-6R, SCF, FGF, IGF-1 and CNTF. The final concentration of each growth factor used at step b) of the above processes is preferably of about 1 ng/m L.

Additionally, in some embodiments, the basal synthetic medium may be supplemented with insulin at the concentration from 1 to 50 mg/L, especially from 1 to 10 mg/L, preferably about 10 mg/L.

Additionally, in some embodiments, the basal synthetic medium may be supplemented with L-glutamine (L-Gln) at the concentration from 0 to 12 mM, preferably from 1 to 5 mM, more preferably about 2.5 mM.

Additionally, in some embodiments, the basal synthetic medium may be supplemented with one or more ingredient(s) selected from the group consisting of amino acids, nucleotides, vitamins, saccharides, fatty acids, beta-mercapto-ethanol, glycine, choline, pluronic acid F-68 and sodium pyruvate.

Additionally, the basal synthetic medium may be supplemented with an animal serum (e.g., fetal calf serum) at the concentration from 1% to 10%. Preferably, the animal serum concentration at step b) of the above processes is of about 5 to 10%. In some embodiments, a serum-free basal culture medium is used.

Alternative to the animal serum, a protein hydrolysate of non-animal origin may be used to complement the basal medium. Protein hydrolysates of non-animal origin are selected from the group consisting of bacteria tryptone, yeast tryptone, yeast or plant hydrolysates, such as soy hydrolysates, or a mixture thereof. In a preferred embodiment, the protein hydrolysate of non-animal origin is soy hydrolysate.

For the establishment of the avian cell line of the invention, the preferred basal medium is DMEM-HamF12 medium complemented with 2 mM L-glutamine, 1 mM sodium pyruvate, 1% non-essential amino acids, vitamins 1%, 0.16 mM beta-mercapto-ethanol, and optionally with 1× yeast hydrolysate.

More details on the conditions used for the establishment of the avian cell line can be found in WO2003076601, WO2005007840 and WO2008129058.

In one embodiment, the cell line established in accordance with the above-described methods is the chicken cell line. In another embodiment, the cell line established in accordance with the above-described methods is the duck cell line. The cell lines established in accordance with the above-described methods are genetically stable, continuous, capable to grow in suspension in the basic synthetic medium in the absence of exogenous growth factors, feeder cells and/or animal serum. They also exhibit sustained viability and replicative capacity in long-term culture conditions, and therefore are ideally suited to be grown on an industrial scale for producing a high yield biomass usable as food.

In another embodiment, the avian cell line of the present invention is selected from, but not limited to, the avian EBx cell lines already described in the patent applications WO2003076601, WO2005007840 and WO2008129058, provided that the cell line has all characteristics as described above. Accordingly, the cell line of the present invention may be the chicken cell line, especially non-adherent chicken cell line selected from the group consisting of EB1, EB3, EB4, EB5, EB14, EB line 0 and EBv13 cell lines (described in WO2003076601 and WO2005007840). Preferably, the chicken cell line is free of infectious endogenous retroviruses as EB line 0, or the chicken cell line is derived from SPF species as EBv13, both described in WO2008129058. Most preferably, the chicken cell line is the cell line derived from EBv13, in particular DL43 and DL46, obtained by the process of the aspect A3 described above in the Summary of the Invention.

According to a preferred embodiment, the cell line may be any duck EBx cell line described in WO2008129058. Particularly, the duck cell line may be selected from the group consisting of, but not limited to, EB24, EB26 and EB66 cell lines. Most preferably, the duck cell line is EB24 (WP24) or EB26 (WP26). The cell line names EB24 and WP24, as well as EB26 and WP26, as used in this application, are interchangeable. All duck EBx cell lines have common features: they derive from duck ES cells, are stable, continuous, can grow in high-density suspension in the synthetic medium in the absence of exogenous growth factors, feeder cells and/or animal serum over a long period or indefinitely. Importantly, they do not comprise ALV-E and/or EAV proviral sequences in their genomes and therefore are free of endogenous replication-competent retroviral particle.

In yet one embodiment, the cell line of the invention is a new avian cell line obtained by one of the processes described above, wherein said cell line is characterized in that it is stable, continuous, free of any endogenous or exogenous virus particle, genomic proviral and/or tumorigenic sequence, capable of proliferating in a basic synthetic medium in the absence of additional growth factor(s), such as natural or synthetic hormones or their derivatives, feeder cells and/or any additional animal product (including serum), can grow in suspension until a high cell density and produce high yield biomass.

Alternatively, the avian cell line may be selected from any commercially available cell lines including, but not limited to, duck cell line AGE1.CR®.pIX (described in WO2005042728), DuckCelt®-T17 cell line (described in WO2009004016) and quail cell line QOR/2E11 (described in WO2009137146). Briefly, AGE1.CR®.pIX is the genetically modified duck cell line derived from retina or embryonic fibroblasts immortalized by transfection of adenovirus genes. Another genetically modified duck cell line DuckCelt®-T17 was generated from primary embryonic cells of Cairina moschata by integration into genome of E1A sequences. The quail QOR/2E11 cell line was obtained from quail embryos by UV-irradiation as an adherent cell line, but adaptation to grow in suspension was also reported (see Kraus et al., 2011).

Avian cell lines of the invention may be further characterized by standard methods known in the art. For instance, a potential way of characterizing and determining specific feature(s) of a cell line may be the sequencing of the genome of said cell line. Once a complete genome is known, a copy of the cell line may be obtained by starting with a cell line of very similar genomic sequence and then altering the sequence by gene editing, such as the CRISPR-Cas 9 method (see Hsu et al., 2014).

### Process of Producing Avian Cell Biomass

In another aspect, the present application provides the process of scaled-up and high-yield production of cell biomass derived from the avian cell line described above. Briefly, this process includes, but is not limited to, the following steps: adapting cells from a master or working bank to a cell culture medium; scaling up the adapted cell sub-culture in various size T-flasks or Erlenmeyers, seeding a suitable bioreactor with the adapted cells; culturing suspension of the adapted avian cells in a synthetic culture medium until a high density of cells will be reached; and harvesting cell biomass by filtration, or centrifugation, or precipitation (sedimentation and decantation), or any kind of methods permitting to separate cells from the medium.

It should also be noted that variations of the above-mentioned process that would give rise to production of the large cell biomass are also encompassed by the present invention.

The present application also provides conditions for the large-scale production of the avian cell biomass.

In particular, the application provides the cell culture medium, which is a synthetic medium free of substances hazardous for humans and/or animals. More specifically, the medium may be selected from the group including, but not limited to, BME (basal Eagle Medium), MEM (minimum Eagle Medium), medium 199, DMEM (Dulbecco's modified Eagle Medium), GMEM (Glasgow's modified Eagle medium), DMEM-HamF12, Ham-F12, Ham-F10, IMDM (Iscove's Modified Dulbecco's medium), MacCoy's 5A medium, RPMI 1640, GTM3, Ex-Cell® EBX™ GRO-I, HYQ CDM4 PermAb and HYQ CDM4 Avian medium (Hyclone), L-15 (Leibovitz), OptiPRO™ SFM and 293 SFM II, or combinations thereof. Alternatively, the culture medium may be a new synthetic medium developed experimentally, for instance, by combination or modification of the commercial mediums. In order to improve cell growth, additional ingredients may be added to the medium. They include, but are not limited to, amino acids (nonessential or essential amino acids), especially L-glutamine, methionine, glutamate, aspartate, asparagine, nucleotides, insulin, vitamins (e.g. thiamine, riboflavin, folic acid, D-Ca panthothenate), saccharides (e.g. D-glucose, D-sucrose, D-galactose or mixtures thereof), fatty acids, beta-mercapto-ethanol, glycine, choline, pluronic acid F-68 and sodium pyruvate. The final concentration of L-glutamine (L-Gln) in the culture medium may be used in the range from 0 to 12 mM or from 0 to 10 mM, especially from 1 to 5, more especially from 2 to 4 mM, preferably about 2.5 mM. The final concentration of insulin in the medium may be in the range from 1 to 50 mg/L, especially from 1 to 10 mg/L, preferably about 10 mg/L.

Preferably, the culture medium is free of any animal product, especially free of an animal serum. “Serum-free medium” (SFM) meant a cell culture medium ready to use, that does not required animal serum. The SFM medium of the invention comprises a number of ingredients, including amino acids, vitamins, organic and inorganic salts, sources of carbohydrate, each ingredient being present in an amount, which supports the cultivation of a cell in vitro. This medium is not necessarily chemically defined, and may contain hydrolysates of various origin, from plant (e.g., soy) or yeast for instance. In a preferred embodiment, the culture medium is the chemically defined SFM that does not contain components of animal or human origin (“free of animal origin”).

Preferably, the cell culture is carried out in HYQ CDM4 Avian medium or a combination thereof, especially in HYQ CDM4 Avian medium supplemented with L-Gln used at the concentration from 2.5 to 4 mM.

According to another embodiment of the present invention, the cells are grown in suspension without any support or matrix. Alternatively, the cells may be attached to a substrate, attached to a scaffold or attached to microcarrier beads or gels.

According to other embodiments of the present invention, the cell culture may be performed in batch, fed-batch, perfusion, or continuous mode.

Briefly, fed-batch culture is, in the broadest sense, defined as an operational technique in biotechnological processes where one or more nutrients are fed to the bioreactor during cultivation and in which the product remain in the bioreactor until the end of the run (Yamanè & Shimizu, 1984). The fed-batch strategy is typically used in bio-industrial processes to reach a high cell density in the bioreactor. Mostly the feed solution is highly concentrated to avoid dilution of the bioreactor, increase of pH and osmolality. The controlled addition of the nutrient directly affects the growth rate of the culture and helps to avoid nutrient depletion, overflow metabolism and oxygen limitation (Jeongseok Lee et al., 1999).

The constantly-fed-batch culture is the one in which the feed rate of a growth-limiting substrate is constant, i.e. the feed rate is invariant during the culture. If the feed rate of the growth-limiting substrate is increased in proportion to the exponential growth rate of the cells, it is possible to maintain exponential cell growth rate for a long time, called exponentially-fed-batch culture.

Perfusion culture means to maintain a cell culture in bioreactor in which equivalent volumes of media are simultaneously added and removed while the cells are retained in the reactor. This provides a steady source of fresh nutrients and constant removal of cell waste products.

The cultivation vessel of the present invention may be selected from, but is not limited to, agitated flask, Erlenmeyer flask, spinner flask, and stirred paddled or wave bioreactors. Particularly, the cultivation vessel may be selected among, but not limited to, continuous stirred tank bioreactor, Wave™ Bioreactor, Belle™ bioreactor, Mobius bioreactor, agitated bioreactor (e.g, Orbshake), bioreactor with perfusion systems. For scaled up production, the preferred cultivation vessel is a bioreactor. The volume of bioreactor may be equal or large than 20 liters, larger than 100 liters, larger than 1,000 liters, preferably up to 10,000 liters. According to the preferred embodiment, the cultivation vessel is a continuous stirred tank bioreactor that allows control of temperature, aeration, pH and other controlled conditions and which is equipped with appropriate inlets for introducing the cells, sterile oxygen, various media for cultivation and outlets for installing probes, removing cells and media and means for agitating the culture medium in the bioreactor.

Typically, cells are scaled-up from a master or working cell bank-vial through various sizes of T-flasks, Erlenmeyer's, roller bottles or Wave™ Bioreactors. The resulting cell suspension is then fed into a larger bioreactor for further cultivation. For example, about 16 billion cells are used to seed the 30 L bioreactor.

In the preferred embodiment of the present invention, the cell culture is carried out at pH 7.2 (regulated with CO₂ or NaOH injection), pO₂ at 50% with the stirring speed at 40 rpm and the temperature at 37° C.

The Population Doubling Time (PDT) in a fed-batch culture may be in the range from 10 to 40 hours, preferably from 10 to 20 hours, more preferably from 10 to 15 hours, most preferably around (or below) 12 hours.

The theoretical maximum cell concentration (cell density), which can be obtained for animal cells in suspension culture, is considered about 10⁹ to 10¹¹ cells/mL. For many of the conventional cell lines used for industrial production, the cell density is in the range of 2×10⁶ to 4×10⁶ cells/mL obtained in fed-batch mode and up to 3×10⁷ cells/mL obtained in perfusion mode (see Tapia et al., 2016).

The avian cell line used in the process of the present invention has high potential for industrial scale production and the selection of the appropriate cell line is important. The main selection criteria is next to the ability to be stable over some passages and be safe to produce biomass in as high amount as possible in the shortest time possible. For example, EB66 cell line can reach the cell density above 1.6×10⁸ cells/mL when cultured in perfusion mode (see Nikolay et al., 2018). Typically, the cell density obtainable for EBx cells in fed-batch culture is in the range from 1×10⁷ to 2×10⁷ cells/mL. In the preferred embodiment, culture cell density reaches about 1×10⁷ cells/mL or more, about 2×10⁷ cells/mL or more, about 5×10⁷ cells/mL or more, about 10⁸ cells/mL or more.

Typically, the cell biomass is in the range from 0.5 to 1.0 mg or more per million cells, preferably from 0.7 to 1.0 mg or more per million cells, more preferably about 1 mg or more per million cells. It is foreseen that the bulk cell yield achievable by the present process may exceed 10¹¹ cells/L.

The typical process of culturing the avian cell suspension comprises the steps:

1) 10 to 20 million of CD medium adapted cells contained in frozen vials are thawed in 37° C. water bath, suspended in about 30 mL of pre-warmed CD medium and placed in an incubator under agitation on a 25 mm orbital throw shaker at 150 rpm, 37° C., 7.5% CO₂ in humidified atmosphere (above 80%),
2) after recovery, the cells of step 1 are sub-cultured and amplified for 3 passages into larger Erlenmeyer flasks seeded at concentration of about from 0.3×10⁶ to 0.5×10⁶ cells/mL. Between each subculture, the Erlenmeyer flasks are incubated at 37° C., 7.5% CO₂ and 150 rpm for 3 days.
3) after 3 passages, the cells are seeded in a 30 L bioreactor in 20 L of CD medium at a volume ratio of around 1:10; the cells are cultured during 3 days at 37° C., 40 rpm, 50% O₂ until a cell density of at least 10⁷ cells/mL is reached.
4) the cells are harvested by centrifugation at 3450 g for 10 min, or by filtration, or by precipitation.

In one embodiment, cell precipitation may be performed by adding to cell suspension the calcium salt. The calcium salt may be selected from the group consisting of, but not limited to, calcium chloride, calcium acetate, calcium carbonate, calcium citrate and calcium lactate. Preferably, calcium chloride is used. The final concentration of the calcium chloride is in the range from 10 to 500 mg/L, preferably from 50 to 300 mg/L, more preferably is 50 mg/L. After addition of calcium chloride, avian cells form large aggregates (clumps) which will precipitate. Calcium chloride may be added to a bioreactor at the end of cell amplification process. As the result, cell biomass will be sediment in the bottom of the container and the supernatant can be removed by decantation. If the harvesting ports are located at the lowest part of the containers, the concentrated cell “paste” in a reduced volume can be collected and used in the next steps of the bioprocess.

An example of perfusion cultivation of the avian EBx cell line, particularly EB66 cell line, in a bioreactor, is described in Nikolay at el., 2018. Briefly, 1 L bioreactors were operated with scalable hollow fiber-based tangential flow filtration (TFF) and alternating tangential flow filtration (ATF) perfusion systems.

Culturing in a perfusion bioreactor was performed at fixed cell-specific perfusion rate (CSPR) calculated as CSPR=D_{perf}/Xᵥ, wherein D_{perf} is perfused media volume, and Xᵥ is viable cell concentration. CSPRs can vary strongly between bioprocesses and are typically chosen in the range of 50-500 pL/cell/day depending on the feeding profile (Konstantinov et al., 2006). Growing EB66 cells in the chemically defined CDM4Avian medium at CSPR of 34 pL/cell/day resulted at the cell concentration of 1.6×10⁸ cells/mL. In another example of perfusion, cultivation of AGE1.CR.pIX® cell line conducted in manual mode at CSPR of about 60 pL/cell/day, the cell concentration of 5.0×10⁷ cells/mL was achieved (Vázquez-Ramírez et al., 2018).

In a preferred embodiment of the invention, aseptic techniques have to be used for culturing the avian cells and preparing final food products that are substantially free from hazard microbes, such as bacteria, fungi, viruses, prions, protozoa, or any combination of the above. Preferably, the production is conducted under Good Manufacturing Practice (GMP) conditions avoiding any harmful contaminations.

In another aspect, the present application provides the cell biomass derived from the avian cell line cultured in vitro. The cell biomass comprises or essentially consists of the avian cells cultured in vitro. The cell biomass may be obtained by the process provided herein or any modified process. Any production process suitable for the avian cell culture may be explored. The high yield cell culture performed in industrial scale is preferred.

In yet another aspect, the present invention relates to use of the avian cell line and the cell biomass described above for production of synthetic food products for human or animal consumption.

In yet another aspect, the present invention provides synthetic food products derived from the avian cells grown in vitro suitable for human or animal consumption.

In one embodiment, the synthetic food product of the invention comprises or essentially consists of the avian cell biomass produced according to any of the processes described above. In one particular embodiment, the synthetic food product comprises or essentially consists of the cell biomass derived from the chicken cell line, preferably the chicken cell line selected from the group consisting of, but not limited to, EB1, EB3, EB4, EB5, EB14, EB line 0 and EBv13, DL43 and DL46 cell lines described above. In another particular embodiment, the cell line may be selected from the group consisting of, but not limited to, duck EB24, EB26 and EB66 cell lines. Alternatively, the synthetic food product may comprises or essentially consists of the cell biomass derived from the avian cell line obtained by any of the processes described herein.

Preferably, the synthetic food product of the present invention comprises or essentially consists of cell biomass obtained from the chicken cell line DL43 or duck cell line EB26 (WP26).

In one embodiment, the synthetic food products of the present invention do not contain any additional component(s) derived from animal origin such as cells, proteins, polypeptides, enzymes, lipids, body fats, animal tissues, serums, etc.

In another embodiment of the invention, the synthetic food products of the invention may further include other cells derived from any animal tissues, such as muscle, fat or cartilage cells, or combinations thereof. These cells may be primary somatic cells derived from any animals such as mammals (e.g. cattle, buffalo, rabbit, pig, sheep, deer, etc.), birds (e.g. chicken, duck, ostrich, turkey, pheasant, etc.), fish (e.g. swordfish, salmon, tuna, sea bass, trout, catfish, etc.), invertebrates (e.g. lobster, crab, shrimp, clams, oyster, mussels, sea urchin, etc.), reptiles (e.g. snake, alligator, turtle, etc.), and amphibians (e.g. frog legs). Alternatively, these cells may be cells derived from pluripotent embryonic stem cells induced into differentiated cells. For instance, muscle cells may be primary muscle cells or may derived from pluripotent embryonic mesenchymal stem cells that give rise to muscle cells, fat cells, bone cells, and cartilage cells. Examples of avian cells include, but are not limited to, the ATTC cell lines DF1 (CRL-12203 chicken), QM7 (quail), DE (duck) and chicken embryonic fibroblasts described in WO2018011805. These cells may be grown in vitro together with the avian cells or added after avian cells harvesting. Addition of those cells may improve taste, aroma and/or nutritional quality of the synthetic meat. For example, fattier meat is tastier and may improve the taste properties of the product. The ratio of meat cells to fat cells may be regulated in vitro to produce the food products with optimal flavor and health effects. Muscle and cartilage cells may improve texture (consistency) of the product. Examples of synthetic food products that have muscle cells and cartilage cells include chicken breast or pork ribs.

In yet another embodiment, other nutrients such as vitamins that are normally lacking in meat products from whole animals may be added to increase the nutritional value of synthetic food. This may be achieved either through straight addition of the nutrients to the growth medium or through genetic engineering techniques. For example, the gene or genes for enzymes responsible for the biosynthesis of a particular vitamin, such as vitamin D, A, or different vitamin B complexes, may be transfected in the cultured avian cells to produce the particular vitamin. Other nutrients include, but are not limited to, essential trace elements, minerals, co-vitamins, essential fatty acids, essential amino acids, enzymes, antioxidants, etc.

In yet another embodiment, the process of the present invention may also include adding a flavorant and/or flavor aromatic. The flavorant may be added during the mixing step, or may be mixed with any of the components (e.g., the cultured cells) before the mixing step. Examples of taste and sensation producing flavorants include artificial sweeteners, glutamic acid salts, glycine salts, guanylic acid salts, inosinic acid salts, ribonucleotide salts, and organic acids, including acetic acid, citric acid, malic acid, tartaric acid, and polyphenolics. A few representative examples of common flavor aromatics include isoamyl acetate (banana), cinnamic aldehyde (cinnamon), ethyl propionate (fruity), limonene (orange), ethyl-(E,Z)-2,4-decadienoate (pear), allyl hexanoate (pineapple), ethyl maltol (sugar, cotton candy), methyl salicylate (wintergreen), and mixtures thereof.

Furthermore, the present invention provides a color enhancer (colorant) which may be added to the cultured cells for making the food product visually more attractive. Additionally, the colorant may function as a physiological antioxidant, thus providing another essential nutrient. For example, colored antioxidants such as some flavonoids, carotenoids, anthocyanins and the like, from tomatoes, black currants, grapes, blueberries, cranberries and the like may be used. Preferably, the colorant is the natural product or the refined or partially refined product. For example, refined catechins, resveratrol, anthocyanin, beta-carotenes, lycopene, lutein, zeaxanthin and the like may be used as the colorant.

In yet another embodiment, the food products of the present invention may be used to generate any kind of food product, where it can contribute to the taste, texture and nutritional content. The synthetic food products of the invention may be pickled, boiled, cooked, smoked, fried, baked, dried or frozen, and typically eaten as a snack or as part of a meal. The final food (edible) products obtained according to the process of the present invention may be configured in any of the consumption forms including, but not limited to, soup, puree, paste, pie, pellets, crumbles, gel, powder, granules, tablet, chips, capsule, spread, sausage, and the like. The final food product can be prepared on 3D printer. 3D printing food is developed by Novameat, Jet-Eat, Meatech and other companies. In particular, Novameat has developed a synthetic, 3D-printed meat with texture of beef or chicken (see https://www.novameat.com/). For a full exploration of 3D food printing (see, e.g., Sun J. et al., 2015).

The final food products each contain some portion of the cultured avian cells as an essential ingredient but may also contain other non-toxic substances, e.g. plant-derived matter (including cultured plant cells).

Finally, it is noted that the above embodiments are only used to illustrate the technical solution of the present invention and are not limited thereto, although reference is made to the above embodiments. The specific implementation manners can be modified or equivalently replaced, but these modifications or changes are not removed from the scope of protection of the claims of the present invention.

### EXAMPLES

### Example 1. Production of Cell Biomass

### Material and Methods

### Cell Bank

An Avian Stem cell bank (Valneva, Duck cell line, GMP Working Cell Bank), prepared from cells adapted to grow in Ex-Cell® EBx™ GRO-I Serum Free Medium (SAFC, ref. 14530C) supplemented with 2.5 mM of L-glutamine (L-Gln), was used as starting material.

The cell line was initially isolated from duck blastoderm and adapted to grow in suspension in the serum free medium without scaffold or matrix. The cells are characterized by their property to grow in suspension without carrier at 37° C. at a small scale (in Erlenmeyer flasks) or at larger scale in bioreactors. Cells proliferate as clumps when maintained under constant agitation.

### CD Growth Medium Preparation

The medium used along the process was the chemically defined medium HYQ CDM4 Avian medium (Hyclone, ref. 5H31036.02) supplemented with 2.5 or 4 mM L-Gln (LONZA, ref. 13E17-605E).

### Freezing Mix

1.46 M sucrose solution was prepared by dissolving 50 g of sucrose powder (Sigma, 51888) in 100 mL of sterile water (B Braun). The solution was then sterile filtered through 0.22 μm filter (Millipore).

The freezing mix contains 20% dimethyl sulfoxide (DMSO) (Sigma, D2438) and 0.2 M sucrose diluted in the fresh CD medium supplemented with 2.5 mM L-Gln. This freezing mix was prepared extemporaneously and placed at 4° C. before use.

### Cell Bank Thawing and CD Medium Adaptation Before Freezing

Cell thawing was performed as quickly as possible by placing the cryovial in a 37° C. water bath. Cells were then diluted in 30 mL of the pre-warmed CD growth medium supplemented with 2.5 mM L-Gln. Cell count and viability were assessed in a cell aliquot with a cell counter based on the trypan exclusion method (VI-Cell XR, Beckman Coulter). To remove the freezing medium, cell centrifugation at 1200 rpm during 10 minutes was applied. After centrifugation, the cell pellet was resuspended in the complete growth medium to get a final seeding concentration comprised between 0.5 to 1.5×10⁶ cells/mL and the cell suspension was transferred into the 125 mL Erlenmeyer flask. The cells were cultured at 37° C. and 7.5% CO₂ at around 90% humidity (Thermo Incubator, Model 311, Hepa Class 100) under constant agitation at 125 rpm (IKA agitator, ref. KS260).

After revitalization, the cell culture was daily checked by microscopic observation. During this post-thawing period, cell counting was regularly done to evaluate cell recovery. Fresh CD growth medium was added at day 2 and day 3 to avoid over density. At day 4, cells were seeded in the 250 mL Erlenmeyer flask at 0.3×10⁶ cells/mL under 60 mL of CD growth medium. Agitation speed was increased to 135 rpm.

For amplification, cells were seeded at 0.3×10⁶ cells/mL in the 500 mL and 1 L Erlenmeyer flasks following supplier recommendation.

### Master Cell Bank (MCB) Freezing

Cells adapted to CD medium were harvested in exponential growth phase in the 500 mL tubes by centrifugation at 1200 rpm during 10 minutes. After centrifugation, the cell pellet was diluted in spent medium at 40×10⁶ cells/mL and an equivalent volume of cold freezing mix was added drop by drop to finally obtain a cell suspension at 20×10⁶ cells/mL. Finally, the cryopreservation medium was composed of DMSO (10%) (Sigma, ref D2438-50 mL), 0.1 M sucrose (6.5%) (Sigma, ref S188), 50% of spent CD medium recovered from the culture and 33.5% of fresh CD medium supplemented with 2.5 mM L-Gln. Cryovials (Corning, ref 430488) were filled with 1 mL of the cell freezing mixture and placed at −80° C. in freezing container (Nalgene, Mr. Frosty™) before transfer in liquid nitrogen (−196° C.) for long term storage.

### Thawing and Culture of CD Medium Adapted Cell Line

Cryovials containing cells adapted to grow in CD medium were thawed in the 125 mL Erlenmeyer flask under 15 mL of fresh CD medium and placed in the shaker incubator (Kuhner, ref ISF1-XC) at 150 rpm agitation speed, 7.5% CO₂ and 80% humidity. After addition of 15 mL and 20 mL of the medium at day 1 and day 2 respectively, cells were sub-cultured at day 3 for further step of amplification.

### Culture at Small Scale

After thawing, cells were grown in the 250 mL to 3 L Erlenmeyer flasks (Corning, Ref 431144, 431147 and 431253) maintained under constant agitation (150 rpm (for 250, 500 or 1 L Erlenmeyer flask) or 80 rpm (3 L Erlenmeyer flasks), 25 mm orbital) in the shaker incubator (Kuhner, ref ISF1-XC) at 37° C., 80% humidity and 7.5% CO₂. Cells were seeded at 0.3×10⁶ cells/mL and were sub-cultured every 3 days. Seeding were performed respectively under 60 mL, 400 mL or 1 L in the 250 mL, 1 L or 3 L Erlenmeyer flasks.

### Growth Kinetics

Cells were seeded in the 250 mL Erlenmeyer flasks at 0.1 to 0.5×10⁶ cells/mL under 100 mL of CD medium supplemented with 2.5 mM L-Gln. After transfer, a daily cell counting was performed to check cell concentration and viability post seeding.

### Parameters Used for Large-Scale Production in 30 L Stirred—Tank Bioreactor

After amplification in the 3 L Erlenmeyer flasks, cells were seeded at 0.8×10⁶ cells/mL in 20 L of pre-warmed medium in a 30 L stainless steel bioreactor (Applikon, Ref ADI 1075). The incubation monitoring was defined as described hereafter: pH 7.2 regulated with CO₂ or NaOH injection, O₂ set point 50%, stirring speed 40 rpm and temperature 37° C. Consumptions of carbon sources (glucose, glutamate and glutamine) and releases of metabolic by-products (lactate and ammonium) were daily monitored along the cell culture (Bioprofile Flex analyzer, Nova Biomedical).

### Cell Harvest and Pellet Preparation

Three days post seeding, cells were collected from the bioreactor in 1 L bottles and submitted to a centrifugation at 3450 g during 10 minutes (Beckman Coulter, Ref AVANTI JXN-26/rotor JL-8.1000). After removal of the spent medium, cells were resuspended in 1×PBS (LONZA, Ref BE17-516F) for rinse and transferred in the 500 mL tubes for a second run of centrifugation at 3450 g (4000 rpm) during 10 minutes (ThermoFisher Scientific, Ref Sorvall ST40). After buffer removal, the 500 mL tubes containing the dry pellets were weighed (Scale: Denver, Ref SI 4002) and placed at −80° C. (Sanyo, Ref MDF-U73V) for storage. The weight of the cell pellet was calculated by subtracting the 500 mL tube weigh to the total weigh (500 mL tube+cell pellet).

### Results

### Avian Stem Cells Adaptation to a Chemically Defined Medium

The first step of the process was the manufacturing of a bank of avian stem cells adapted to grow in the Chemically Defined Medium HYQ CDM4 Avian medium.

The objective of this step was to prepare a unique source of cells:
to avoid several adaptations
to allow possible validation/release of a master cell bank
to use same starting material for several batches of production
to shorten timeline allocated to the chemically defined bio-productions
to minimize batch to batch variability

### Cell Adaptation and Banking

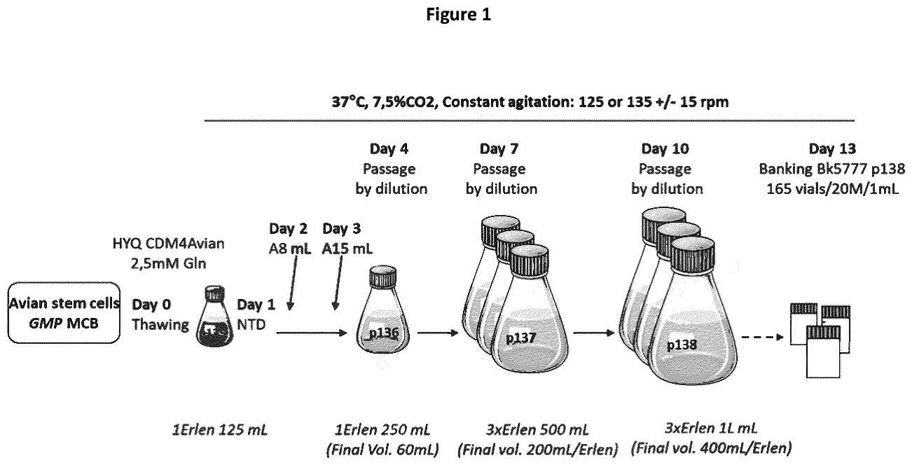

To avoid doing adaptation of stem cells to CD medium for each production round, a single adaptation was conducted and a working bank of 165 vials was prepared as described below and shown in FIG. 1.

One cryovial of the avian stem cells originally grown in Ex-cell GRO-I SFM was thawed directly in 30 mL of CDM4 Avian CD medium supplemented with 2.5 mM of L-Gln. After centrifugation, 7.2×10⁶ cells were recovered and seeded under 12 mL medium at the concentration 0.6×10⁶ cells/mL in a 125 mL-Erlenmeyer. The cells were placed in an incubator on a shaker at 125 rpm. At day 2 and day 3 post thawing, respectively 8 mL and 15 mL of the CD medium were added. At day 4, an aliquot was collected for cell counting and cells were harvested by centrifugation. The cell pellet was resuspended in the fresh CD medium; then one 250 mL-Erlenemeyer was seeded at the concentration 0.3×10⁶ cells/mL under 60 mL and placed in incubation under agitation at 135+/−15 rpm. The next two passages were performed as follows: at day 7 or day 10 cells were harvested and transferred to 3 new 500 mL-Erlenmeyers or 3 L-Erlenmeyers, diluted to 0.3×10⁶ cells/mL under 200 mL or 1 L of the CD medium, respectively. At day 13, around 11 billion cells were collected from the 3 L-Erlenmeyers. The final cell concentration was 9.1×10⁶ cells/mL and viability of 91%.

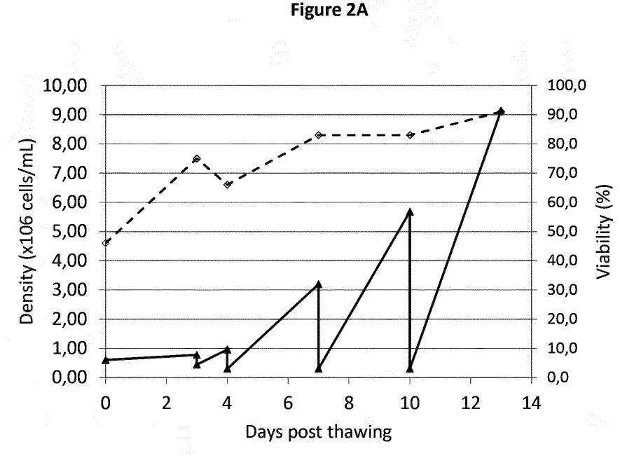

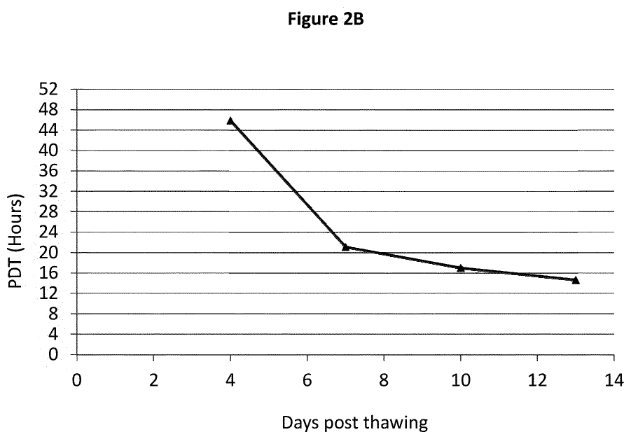

Direct adaptation in HYQ CDM4 Avian medium was very efficient; after 10 days post thawing in the CD medium, cells recovered at expected density of 5×10⁶ cells/mL and good viability (higher than 80%) (see FIG. 2A). In consequence, the Population Doubling Time (PDT), achieved rapidly, was in the expected range of 15 to 16 hours (see FIG. 2B). In terms of morphology, the cells maintained their property to grow in suspension as clumps of dozen of cells, which could be easily resuspended by pipetting. At day 13, cell concentration reached 9.1×10⁶ cells/mL and cell viability 91%. This allowed creating the cell bank of 165 vials (bank 5777). Thus, only four passages and 13 days were enough for adapting the avian stem cells in the HYQ CDM4 Avian CD medium in order to prepare the master cell bank of high quality.

### Validation of Cell Banks

To ensure the quality of the avian stem cell bank after CD adaptation, the cell bank was thawed and cell robustness, viability and stability of cell density and PDT along passages were controlled.

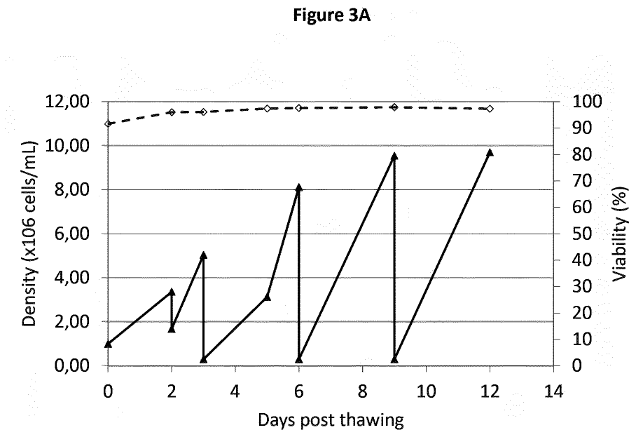

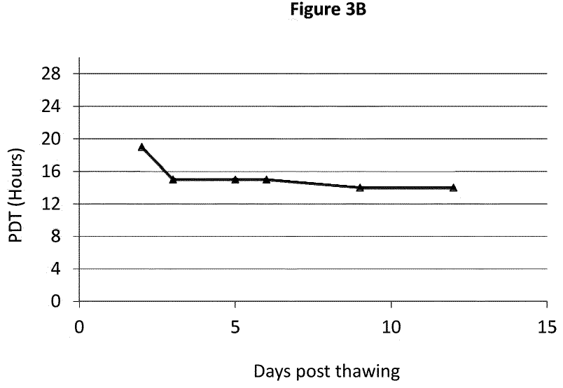

To check cell robustness and stability, the bank 5777 was thawed and maintained in culture during four additional passages. As illustrated in FIG. 3, viability of the bank just after thawing was very good reaching 91%. No cell loss was associated with the freezing step, as the total quantity of cells filled in the vials was fully recovered. After 3 days incubation, the cell density reached around 5×10⁶ cells/mL indicating fast cell proliferation. For the following passages, the concentration higher than 6×10⁶ cell/mL confirmed the good quality of the cell bank.

### Growth Kinetics

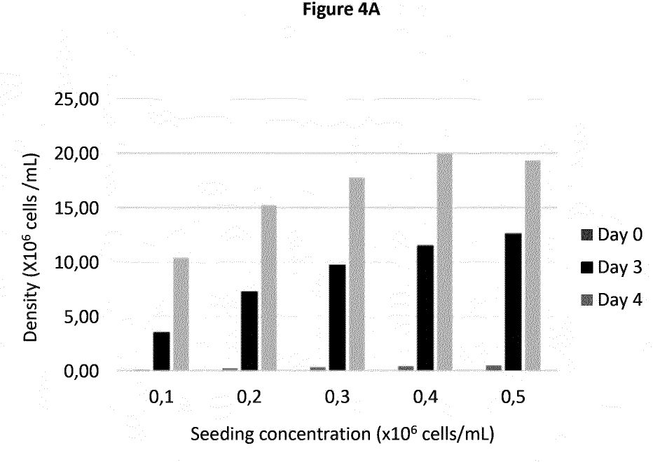

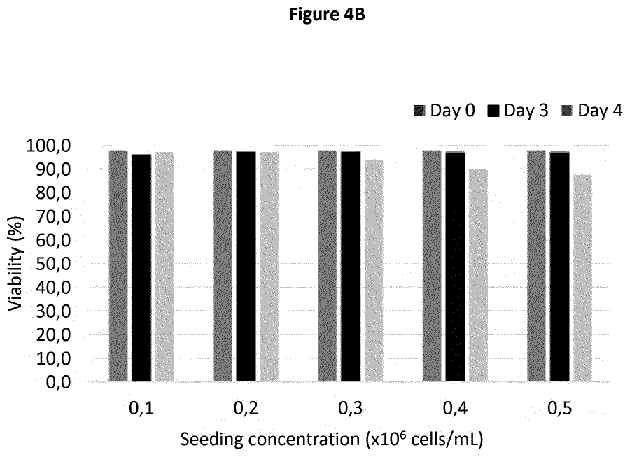

To determine the optimal density that is potentially achievable by adapted avian stem cells, 250 mL-Erlenmeyers were seeded at different concentrations, placed in incubation and daily checked for cell density and viability. FIG. 4A shows cell density obtained after 3 and 4 days of culture; FIG. 4B shows the corresponding cell viability. The data demonstrate that the increase of seeding density up to 0.4×10⁶ cells/mL did not improve optimal cell concentration after 4 days of culture. In all conditions with the seeding higher than 0.2×10⁶ cells/mL, viability tended to decrease slightly at day 4. Regarding viability and cell concentration, a good compromise to reach an optimal density with a good viability in 250 mL-Erlenmeyer would be to seed the cells at the amount of 0.3 to 0.4×10⁶ cells/m L.

### Scale-Up for the Seeding of the 30 L Bioreactor

To produce the cell biomass needed to seed a 30 L bioreactor, we thawed the adapted avian stem cell bank 5777 and amplified cells following a scale-up process performed in Erlenmeyer flasks.

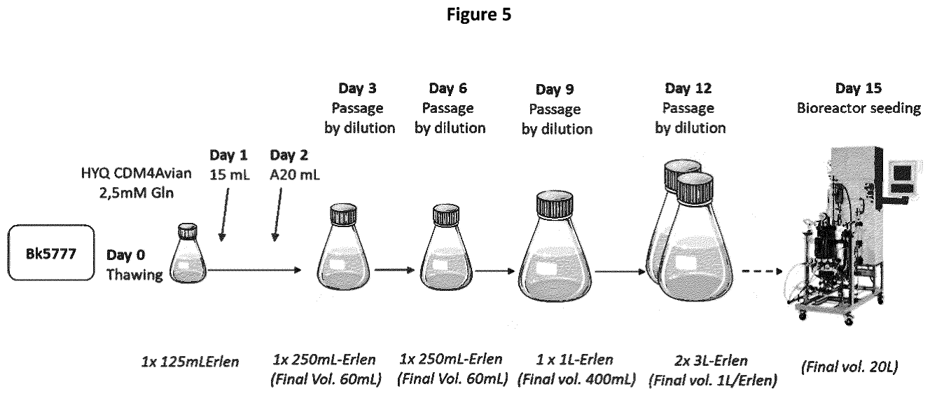

A 30 L stainless steel bioreactor was used for producing the final avian cell biomass in vitro. 16 billion cells were needed to seed the 30 L bioreactor with 20 L of cell suspension at the concentration 0.8×10⁶ cells/mL. Due to the property of the avian stem cells grow at high cell density, the scale-up procedure was not cumbersome as the required amount of cells was obtained only with 2 L suspension. FIG. 5 illustrates a typical process for rapid amplification of the cells for seeding a bioreactor.

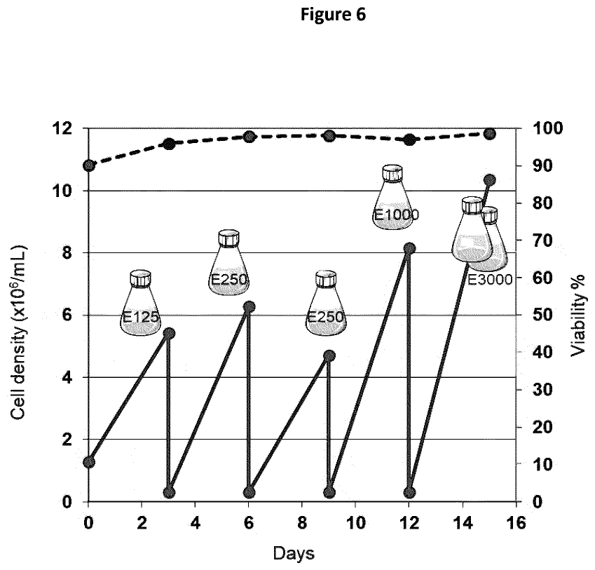

FIG. 6 demonstrates cell densities obtained at each passage along the scale-up process. At the last step of amplification, the achieved cell concentration was 10.3×10⁶ cells/mL, allowing the total harvest of 20.6 billion cells.

### Batch Cell Growth in 30 L Bioreactor

Cells harvested from both 3 L-Erlenmeyers were seeded in the 30 L bioreactor at a concentration of 0.8×10⁶ cells/mL under 20 liters of pre-warmed CD medium supplemented with 4 mM of L-Gln. pH and oxygen regulation set points were adjusted at 7.2 and 50%, respectively, and the agitation rate was 40 rpm. Neither glucose nor glutamine were adjusted as the process was conducted under a batch method. Consumptions of carbon sources (glucose, glutamate and glutamine) and releases of metabolic by-products (lactate and ammonium) were daily monitored along the cell culture (Bioprofile Flex analyzer, Nova Biomedical).

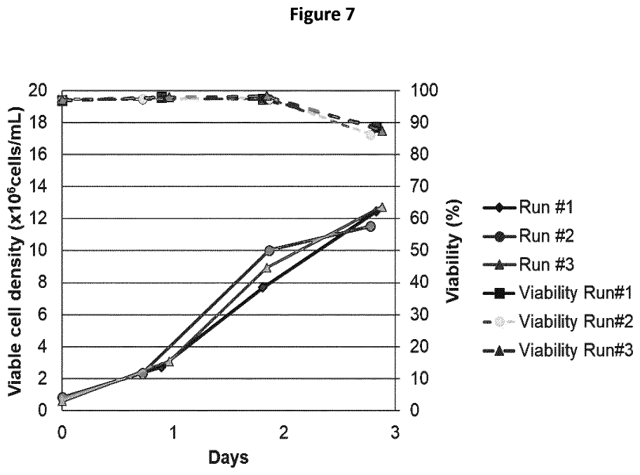

Three runs were conducted using parameters described previously. FIG. 7 illustrates cell growth and viability along the 3 days of production. After seeding, no lag phase was observed and cell proliferation was very fast as shown by the short Population Doubling Time (below 12 hours) between the seeding and day 1 (see Table 1). At day 3, we observed an increase of the PDT (higher than 35 hours) demonstrating a slowdown of proliferation coupled with a decline of viability.

TABLE 1
Follow-up of the Population Doubling
Time after seeding in 30 L bioreactor

| | | |
|---|---|---|
| Run 1 | Run 2 | Run 3 |

A

| | | | |
|---|---|---|---|
| Day 1 | 12 | 11.4 | 9.6 |
| Day2 | 14.7 | 13.1 | 13.7 |
| Day3 | 35.3 | 108.2 | 48.9 |

B

| | | | |
|---|---|---|---|
| Day 1 | 2.7 | 2.4 | 3.1 |
| Day2 | 7.7 | 10 | 8.9 |
| Day3 | 12.5 | 11.5 | 12.7 |

C

| | | | |
|---|---|---|---|
| Day 1 | 98 | 97.4 | 98 |
| Day2 | 97.4 | 97.5 | 98.4 |
| Day3 | 88.7 | 86.3 | 87.5 |

(A) Population Doubling Time (in hours);
(B) viable cell density (in ×10⁶ cells/mL);
(C) viability (in %) along cell production.

Based on the mean of the higher cell concentration obtained for three runs and the corresponding viability, it was concluded that the optimal density was reached between day 2 and day 3 with an approximate concentration of 14×10⁶ total cells/mL.

Metabolite studies conducted during the three runs demonstrated a high consumption of glutamine, glutamate and glucose (data not shown).

### Cell Harvesting

### Centrifugation

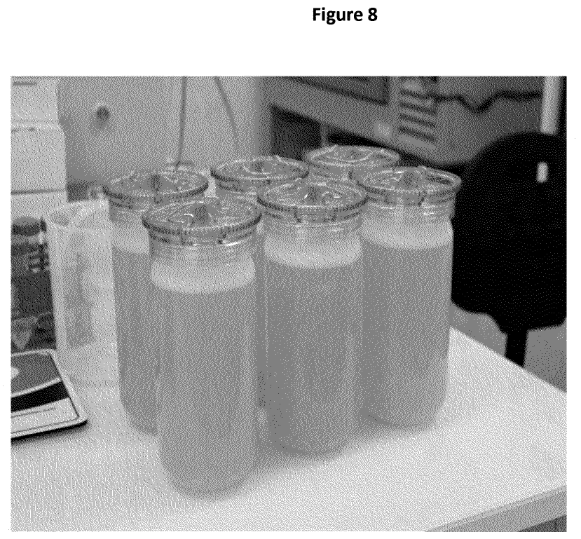

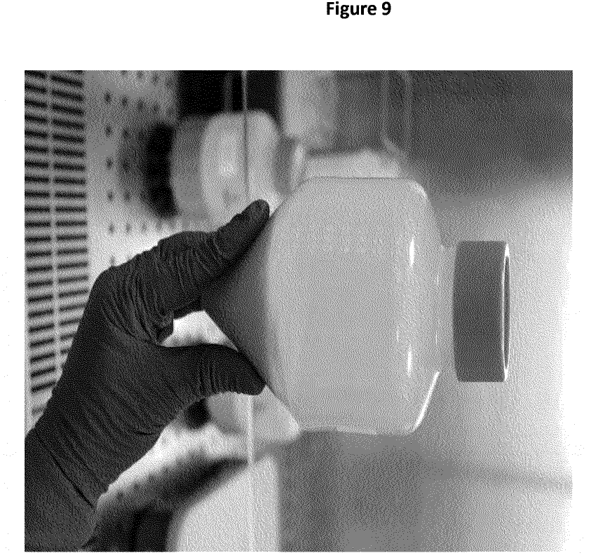

After 3 days of cell growth in the bioreactor, the avian cells were harvested in 1 L-bottles (see FIG. 8) by centrifugation at high speed (3450 g), rinsed in PBS, transferred into 500 mL-tubes and pelleted by a second run of centrifugation (see FIG. 9).

The pellets were weighed after the last run of centrifugation. Respectively, 304 g, 282 g and 281 g were obtained from the run 1, run 2 and run 3 demonstrating the process reproducibility in term of biomass production. Finally, the pellet was frozen at −80° C. for storage.

### Sedimentation and Decantation

The harvest of the cell biomass by centrifugation is a cumbersome process and without a cooling system, an increase of the temperature can be observed after several centrifugation runs with the risk of the alteration of the biological material. So, a step of decantation before centrifugation (or filtration) was considered to reduce the volume of suspension.

As the EBx cells grow as small aggregates, conditions to induce cell clumping were studied to promote the cell sedimentation. Addition of calcium chloride to the medium provokes formation of cell clumps. As duck and chicken cells are not sensitive to the same range of calcium concentrations, different conditions were tested. Chicken or duck cell suspensions at the end of the exponential phase were supplemented with 50, 100, 150, 200 or 300 mg/L of calcium chloride and incubated from 2 to 6 hours at 37° C. under agitation. For EBx cell lines, aggregation was already observed after two hours of incubation. The biggest clumps were produced with the highest calcium concentrations. It was noticed that clump size increased progressively with the calcium concentration. Cell clamping is more pronounced for duck cells as almost all cells are aggregated after 2 hours incubation in the presence of 50 mg/mL of calcium chloride.

To evaluate more precisely the percentage of the cell population sedimented in the bottom of the tubes after 6 hours incubation with calcium chloride and 20 minutes of settling, a cell counting of the residual cells in the supernatants was made. The obtained data are summarized in table 2 and 3. It was observed that 42.8% of the chicken cell suspension can sediment in 20 minutes without calcium addition. The 6 hours treatment improves this percentage of sedimentation with a maximum of 75.5% reached with highest tested dose of calcium chloride (300 mg/L). For the duck cells, no clear sedimentation was observed after 20 minutes without calcium, but addition of 50 mg/L of calcium chloride was sufficient for precipitation of 95% of cell biomass.

Similarly, the step of cell sedimentation could be applied to bioreactors at the end of cell amplification process. As the result, cell biomass will be precipitated in the bottom of the container. If the harvesting ports are located at the lowest part of the containers, the concentrated cell “paste” in a reduced volume can be collected and used in the next steps of the bioprocess.

TABLE 2
Effect of calcium chloride on sedimentation of chicken cells

| Chicken cell Line | |
|---|---|
| Sedimentation time (minutes) | |
| 0 | 20 |

| | CaCl₂ concentration (mg/L) | | | | |
|---|---|---|---|---|---|
| | 0 | 0 | 100 | 200 | 300 |
| Total Cell Density | 8.2 | 4.7 | 2.3 | 2.1 | 2.0 |
| in the supernatant | | | | | |
| (×10⁶ cells/mL) | | | | | |
| Percentage of | 0 | 42.8 | 71.8 | 74.0 | 75.5 |
| sedimentation (%) | | | | | |

TABLE 3
Effect of calcium chloride on sedimentation of duck cell

| Duck cell line | |
|---|---|
| Sedimentation time (minutes) | |
| 0 | 20 |

CaCl₂ concentration (mg/L)

| | 0 | 0 | 50 | 100 | 150 |
|---|---|---|---|---|---|
| Total Cell Density | 17.0 | 16.9 | 0.8 | 0.5 | 0.5 |
| in the supernatant | | | | | |
| (×10⁶ cells/mL) | | | | | |
| Percentage of | 0 | 0.9 | 95.4 | 97.0 | 97.2 |
| sedimentation (%) | | | | | |

Other calcium salts, such as calcium acetate, calcium carbonate, calcium citrate and calcium lactate or alike, may be considered as alternatives.

### Production Yield

Run 1, run 2 and run 3 produced respectively 304 g, 282 g and 281 g of avian stem cells. So, based on cell quantity harvested from the bioreactors (see Table 2), the biomass productivity (total weigh divided by total cell harvested) was 1.18+/−0.07 mg per million cells. As 385.6 g of the medium powder was necessary to conduct 20 L bioreactor, the production yield was about 0.75 g biomass per g medium powder.

TABLE 4
Production yields and productivity

| | Run1 | Run 2 | Run 3 | Average |
|---|---|---|---|---|
| Day3 density | 12.5 | 11.5 | 12.7 | 12.3 |
| (×10⁶ cells/mL) | | | | |
| Total cell | 250 | 230 | 254 | 245 |
| harvested | | | | |
| (×10⁹ cells) | | | | |
| Biomass weigh | 304 | 282 | 281 | 289 |
| (g) | | | | |
| mg of | 1.22 | 1.23 | 1.1 | 1.18 |
| cells/×10⁶ cells | | | | |
| Ratio | 0.79 | 0.73 | 0.73 | 0.75 |
| (g of biomass/g of | | | | |
| medium powder) | | | | |

So, based on the data obtained during the kinetics in Erlenmeyers and the metabolite consumption, improvement of the product yield could be achieved by:
modifying initial cell seeding to extend the cell growth after day 3;
supplementing the CD medium to avoid depletion;
applying the fed batch or perfusion process.

### LIST OF REFERENCES

Biswas and Hutchins. 2007. “Embryonic Stem Cells.” Stem cells and Development 16:213-221.
Chang et al., 1992. “Simple method for isolation of primordial germ cells from chick embryos.” Cell Biol Int. Reports 16(9):853-857.
Eyal-Giladi and Kochan, 1976. “From cleavage to primitive steak formation: a complementary normal table and a new look at the first stage of the development of the chick.” Developmental Biology 49:321-337.
Galbraith H. 2002. “Hormones in international meat production: biological, sociological and consumer issues.” Nutrition Research Reviews 15:293-314.
GEAS. October 2012. “Growing greenhouse gas emissions due to meat production.”
Greger M. 2007. “The human/animal interface: emergence and resurgence of zoonotic infection diseases”. Critical Reviews in Microbiology 33:243-299.
Hamburger V. and Hamilton H. 1951. “A series of normal stages in the development of the chick embryo”.
Hedenus F., Wirsenius D. and Johansson J. A. 2014. “The importance of reduced meat and dairy comsimption for meeting stringent climate change targets”. Climatic Change 124:79-91.
Hsu P., Lander E. S., and Zhang F. 2014. “Development and application of CRISPR_Cas9 for genome engineering”. Cell 157, June 5:1262-1278.
Jeong SH. et al., 2010. “Risk assessment of growth hormones and antimicrobial residues in meat:” Toxicol. Res. 26(4):301.313.
Jeongseok Lee et al. 1999. “Control of fed-batch fermentations.” Biotechnol Adv 17:29-48.
Kadim I. T. et al. 2015. “Cultured meat from muscle stem cells: a review of challenges and prospects.” J. Integrative Agriculture 14(2):222-233.
Konstantinov K. et al. 2006. “The “push-to-low” approach for optimization of high-density perfusion cultures of animal cells.” Adv Biochem Engin Biotechnol 101:75-98.
Kraus B. et al. 2011. “Avian cell line—technology for large scale vaccine production.” BMC Proceedings 5(suppl 8):52.
Mekonnen and Hoekstra. “The green, blue and gray water footprint of farm animals and animal products.” UNESCO-IHE, Research Report Series No. 48. December 2010.
Naito M. et al. 1994. “Production of germline chimeric chickens, with high transmission rate of donor-derived gametes, produced by transfer of primordial germ cells.” Molecule Reproduction and Development 39:153-161.
Nikolay A. et al. 2018. “Process intensification of EB66 cell cultivations leads to high-yield yellow fever and Zika virus production.” Applied Microbiol Biotechnol 102:8725-8737.
Olivier S. et al. 2010. “EB66 cell line, a duck embryonic stem cell-derived substrate for the industrial production of therapeutic monoclonal antibodies with enhanced ADCC activity.” MAbs 2(4):405-15.
Opio C., Gerber P., Falcucci A., et al., 2013. “Greenhouse gas emissions from ruminant supply chains. A global life cycle assessment.” Report prepared by Food and Agriculture organization of the United Nations (FAO), Rome.
Resnick R. et al. 1990. “Phylogenetic Distribution of the Novel Avian Endogenous Provirus Family EAV-0.” L Virology 64(10):4640:4653.
Ritchie and Roser. 2018. “Meat and Seafood Production & Consumption.”
Sellier N., Brillard J-P and Bakst M. R. 2006. “Comparative staging of embryo development in chicken, turkey, duck, goose, Guinea fowl, and Japanese quail assessed from five hours after fertilization through seventy-two hours of incubation.” J Appl Poult Res 15:219-228.
Sun J. et al. 2015. “A Review on 3D Printing for Customized Food Fabrication”, Procedia Manufacturing 1: 308-319.
Tapia F. et al. 2016. “Bioreactors for high cell density and continuous multi-stage cultivations: options for process intensification in cell culture-based viral vaccine production.” Appl Microbiol Biotechnol 100:2121-2132.
Tuomisto H, Ellis M. J. and Haastrup P. “Environmental impacts of cultured meat: alternative production scenarios.” Proceedings of the 9th International Conference on Life Cycle Assessment in the Agri-Food Sector. 2014
Vázquez-Ramírez D. et al. 2018. “High-cell-density cultivations to increase MVA virus production.” Vaccine 36:3124-3133.
Yamanè & Shimizu. 1984. “Fed-batch Techniques in Microbial Processes.” Advances in Biochem Eng. Biotechnol 30:147-194.
Yasuda Y. et al. “A method to obtain avian germ-line chimaeras using isolated primordial germ cells.” J Reprod. Fert. 96:521-528. 1992.

1. A method for in vitro producing a nutritive food product for human or animal consumption comprising culturing an avian cell line in suspension, wherein said avian cell line is i) derived from avian embryonic stem cells, ii) capable of proliferating in a basal culture medium in the absence of exogenous growth factors, feeder cells and/or animal serum, and iii) capable of growing continuously in suspension.

2. The method of claim 1, wherein the avian cell line is obtained by the process comprising the steps:
a) isolating avian embryonic stem cells from an embryo(s) at a developmental stage around oviposition;
b) culturing said cells in a basal culture medium containing at least one exogenous growth factors SCF, IGF-1, bFGF, IL-6, IL-6R and/or CNTF, a layer of feeder cells and an animal serum for at least twenty passages;
c) modifying said culture medium by progressive deprivation of said growth factors, feeder cells and an animal serum and further culturing the cells for at least several passages; and
d) adapting the cells of step c) to suspension,
thereby obtaining the established avian cell line capable of proliferating in a basal culture medium in the absence of exogenous growth factors, feeder cells and/or animal serum for at least 50 days.

3. The method of claim 1, wherein the avian cell line is obtained by the process comprising the steps:
a) isolating the avian embryonic stem cells from an embryo(s) at a developmental stage around oviposition;
b) culturing said cells in a basal culture medium containing the exogenous growth factor IGF-1 and CNTF, a layer of feeder cells and an animal serum for at least one passage;
c) progressively withdrawing said growth factors from the culture of step b) and further growing for at least one passage;
d) progressively withdrawing the feeder cells from the culture of step c) and further growing for at least one passage;
e) progressively withdrawing the animal serum from the culture of step d) and further growing for at least one passage; and
f) adapting the cells of step e) to suspension,
thereby obtaining the continuous avian cell line capable of proliferating in a basal medium in the absence of exogenous growth factors, feeder cells and/or animal serum.

4. The method of claim 1, wherein the avian cell line is derived from a chicken embryonic stem cell.

5. The method of claim 1, wherein the avian cell line is derived from a duck embryonic stem cell.

6. The method of claim 1, wherein the avian cell line is free of functional endogenous retroviral or other viral particles.

7. The method of claim 1, wherein the avian cell line is derived from a specific-pathogen-free (SPF) specie.

8. The method of claim 1, wherein the avian cell line is selected from the group consisting of the chicken EB14, chicken EB line 0, chicken EBv13, chicken DL43, chicken DL46, duck EB24, duck EB26 and duck EB66 cell lines.

9. The method of claim 1, wherein the avian cell line is selected from the group consisting of the chicken DL43, chicken DL46, duck EB24 and duck EB26 cell lines.

10. The method of claim 1, wherein the avian cell line is the chicken DL43 or duck EB26.

11. The method of claim 1, wherein the cell line is grown in a basal culture medium is a synthetic or chemically defined (CD) medium free of hazardous substances for humans and/or animals and free of any animal product, including serum.

12. The method of claim 1, wherein the synthetic medium is Ex-Cell® GRO-I and/or HYQ CDM4 Avian medium.

13. The method of claim 1, wherein the basal culture medium is additionally supplemented with one or more ingredient(s) selected from the group consisting of amino acids, nucleotides, vitamins, saccharides, fatty acids, beta-mercapto-ethanol, insulin, glycine, choline, pluronic acid F-68 and sodium pyruvate, and wherein the basal culture medium optionally further comprises plant and/or yeast hydrolysate.

14. The method of claim 1, wherein the basal culture medium is additionally supplemented with L glutamine used at a concentration from 0 to 12 mM, from 1 to 5 mM, or about 2.5 mM.

15. (canceled)

16. (canceled)

17. (canceled)

18. (canceled)

19. (canceled)

20. (canceled)

21. (canceled)

22. (canceled)

23. (canceled)

24. The method of claim 1, wherein the method yields at least about 0.5 to 1 g biomass per g medium.

25. The method of claim 1, further comprising a step of harvesting cell biomass by sedimentation and decantation, performed by the addition of calcium chloride used at final concentration from 10 to 500 mg/L, from 50 to 300 mg/L, or 50 mg/L.

26. (canceled)

27. (canceled)

28. The method of claim 25, further comprising a step of adding to the cell biomass one or more ingredient(s) increasing nutritional value of the food product or adding flavor or color, comprising vitamins, co-vitamins, minerals, essential amino acids, essential fatty acids, enzymes, antioxidants, flavorant(s), flavor aromatic(s) and/or colorant(s).

29. (canceled)

30. (canceled)

31. A nutritive food product for human or animal consumption produced by the method of claim 1.

32. (canceled)

33. (canceled)

34. (canceled)

35. The nutritive food product of claim 31, further comprising other cells such as non-human muscle cells, fat cells or cartilage cells, or their combinations, that are grown in vitro together with the avian cells or added after harvesting the avian cells.

36. (canceled)

37. (canceled)

38. The nutritive food product of claim 31, wherein the food product is processed to any of the consumption form selected from the group comprising paste, puree, soup, pie, powder, granules, chip, tablet, capsule, spread and sausage.
